(19) United States
(12) Patent Application Publication
(10) Pub. No.: US 2019/0234922 A1
(43) Pub. Date: Aug. 1, 2019

(54) HYDROGEN SULFIDE DETECTING APPARATUS

(71) Applicant: Board of Supervisors of Louisiana State University and Agricultural and Mechanical College, Baton Rouge, LA (US)

(72) Inventors: Christopher KEVIL, Shreveport, LA (US); John CHIDLOW, Shreveport, LA (US); Kevin PAVLICK, Shreveport, LA (US); Suvhashis THAPA, Shreveport, LA (US)

(21) Appl. No.: 16/377,738

(22) Filed: Apr. 8, 2019

Related U.S. Application Data
(63) Continuation-in-part of application No. 14/780,799, filed on Sep. 28, 2015, now Pat. No. 10,254,262.
(60) Provisional application No. 61/806,017, filed on Mar. 28, 2013.

(51) Int. Cl. G01N 33/00

(52) U.S. Cl. CPC G01N 33/0044; G01N 33/0013

(57) ABSTRACT

Methods and hydrogen sulfide (H₂S) detecting apparatuses comprising a single reaction chamber defining a first volume, a single trapping chamber positioned adjacent to the reaction chamber defining a second volume, and an H₂S-permeable membrane positioned between and separating the reaction chamber and the of trapping chamber, wherein the first volume is greater than the second volume.

### CROSS REFERENCE TO RELATED APPLICATIONS/PRIORITY

The present invention is a continuation in part of and claims priority to U.S. patent application Ser. No. 14/780,799 filed Sep. 28, 2015, which claims priority to U.S. Provisional Patent Application No. 61/806,017, filed Mar. 28, 2013, both of which are incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### FIELD OF THE INVENTION

The present invention relates to novel apparatuses and methods for the measurement of hydrogen sulfide, including in some embodiments in its various bioavailable forms.

### BACKGROUND OF THE INVENTION

Hydrogen sulfide (H₂S) is a colorless gasotransmitter (gaseous signaling molecule) that plays a vital role in numerous cellular functions within the human body. For instance, over the past decade, the role of H₂S beyond a toxicant and environmental pollutant has evolved to encompass several biochemical functions that are important in various physiological and pathological responses such as cardiovascular (dys)function, neurological (dys)function, gastrointestinal (dys)function, immune (dys)function, and several other molecular and cell biology responses.

For instance, H₂S has been discovered to have significant potential to contribute to the detection and treatment of cardiovascular disease, including atherosclerosis and peripheral arterial disease. Because of decreased oxidative modification of low-density lipoprotein (LDL), H₂S has also been shown to play a significant role in atherosclerosis by having noted effects on monocyte recruitment, transformation into tissue macrophages, and foam cell formation. Further, H₂S has been shown to inhibit hypochlorite and hemin-mediated atherogenic modification of LDL. Plasma H₂S levels have also been shown to be lower in atherosclerotic plaque, and treatment with sodium hydrosulfide (NaHS) decreases both aortic plaque and intercellular adhesion molecule-1 (ICAM-1). In addition, H₂S down regulates the expression of monocyte chemoattractant protein-1, a CC chemokine that binds to the C—C chemokine receptor type 2 (CCR2) and recruits monocytes into the subendothelial layer to form atherosclerotic plaque. Another critical role of H₂S in the pathogenesis of atherosclerosis is the effect of inducing apoptosis on vascular smooth muscle cells, which generates atherosclerotic plaque. Hydrogen sulfide, administered as NaHS, decreases the proliferation of vascular smooth muscle cell via a mitogen-activated protein kinase (MAPK) pathway in a dose-dependent fashion in rat models. Additional work with a rat model reveals that H₂S reduced vascular calcification. Additionally, recent studies have shown H₂S to have a direct relationship with nitrogen monoxide and carbon monoxide in peripheral arterial disease (PAD) identification.

Hydrogen sulfide arises from multiple biological sources and tissues (e.g. bacteria and organ-specific production). Endogenous biological H₂S production primarily originates from cysteine metabolism through the activity of cystathionine β-synthase and cystathione γ-lyase or through 3-m ercaptopyruvate metabolism by 3-mercaptosulfurtransferase. H₂S can come from redox-dependent metabolism of polysulfides involving glutathione or other small molecular weight thiol modifiers. Lastly, H₂S also arises from different environmental sources that affect humans such as petroleum production and exploration, food and beverage processing, waste disposal and sewage treatment, agriculture and farming, and bacterial contamination and function.

Hydrogen sulfide chemistry is complex and plays several roles in modulating protein thiol function. It affects numerous biological responses involving signal transduction responses, mitochondrial respiration, gene expression, and cell survival/viability. At a physiological pH of ⁻7.2-7.4, H₂S predominantly (^{˜}80%) exists in its anion HS⁻ form with a smaller amount in the gaseous H₂S form (^{˜}20%). This is due to pKa regulation of H₂S forms in aqueous solutions as illustrated in the following equation:

pKa₁=7.04 pKa₂≥13

H₂S⇄HS⁻⇄S²⁻

Due to the different pKa's, the ionic distribution is easily manipulated and, in turn, its distribution controlled in either aqueous or gas phases.

Hydrogen sulfide is very reactive within biological or environmental systems, resulting in sulfide equivalents being present in three different volatile sulfur pools as shown in FIG. 1. These three pools—free H₂S, acid labile H₂S, and sulfane sulfur species—are important in regulating the amount of bioavailable sulfur. Free hydrogen sulfide is found dissolved in plasma and other tissue fluids. At mammalian body conditions (i.e., pH 7.4 and temperature of 37° C.), 18.5% of free hydrogen sulfide exists as H₂S gas, and the remainder is almost all hydrosulfide anion (HS−) with a negligible contribution of S^{2′}. Sulfane sulfur refers to divalent sulfur atoms bound to another sulfur, though they may bear an ionizable hydrogen at some pH values. Examples of these bound sulfurs include thiosulfate S₂O₃²⁻, persulfides R—S—SH, thiosulfaonates R—S(O)—S—R′, polysulfides R—Sₙ—R, polythionates SnO₆²⁻, and elemental sulfur S. Acid labile sulfide, the other major bioavailable pool, consists of sulfur present in iron-sulfur clusters contained in iron-sulfur proteins (non-heme), which are ubiquitous in living organisms, and include a variety of proteins and enzymes, including without limitation, rubredoxins, ferredoxins, aconitase, and succinate dehydrogenase. The acid labile sulfides readily liberate free H₂S in acid conditions (pH<5.4), and the process of acid liberation may also release hydrogen sulfide from persulfides, which have traditionally been classified as sulfane sulfur. This acid labile sulfur pool has been postulated to be a reversible sulfide sink and may be an important storage pool that regulates the amount of bioavailable free hydrogen sulfide.

H₂S equivalents are readily mobilized from these pools based on changes in pH, O₂ concentration, and oxidative/reductive chemistry that affect biological and biochemical responses. Thus, detection of H₂S availability from these distinct pools is important for clinical pathophysiology diagnosis, environmental source identification, and any other organic or inorganic chemistry uses.

Unfortunately, a significant barrier to the study of hydrogen sulfide's role in human health and disease has been the lack of precise methodology and testing means for the accurate and reproducible measurement of hydrogen sulfide both in vivo and in vitro. A variety of methods to measure free H₂S have been employed, but with divergent results. These methods include a spectrophotometric derivatization method resulting in methylene blue formation, variations of this methylene blue method using high performance liquid chromatography, sulfide ion-selective electrodes, polarographic sensors, gas chromatography, and high-performance liquid chromatography (HPLC) in conjunction with fluorimetric based methods using monobromobimane (MBB) to derivatize free H₂S. The complexity of analytical H₂S measurement, especially in living organisms, reflects the fact that hydrogen sulfide is a reactive gas and exists in the organism in the three different volatile sulfur pools shown in FIG. 1. Due to a lack of reliable, accurate analytical detection methods available to quantify H₂S and its various forms, there is great disagreement regarding precise amounts and sources of H₂S metabolism in biological and biochemical settings. Therefore, there is a great need for an apparatus and associated methodology that can be used to accurately and conveniently measure H₂S in its various bioavailable forms.

### SUMMARY OF THE INVENTION

Wherefore, it is an object of the present invention to overcome the above mentioned shortcomings and drawbacks associated with the current technology.

The invention disclosed herein is directed to a hydrogen sulfide (H₂S) detecting apparatus for measuring the concentrations of hydrogen sulfide species in a given sample. In a particular embodiment exemplifying the principles of the invention, the hydrogen sulfide detecting apparatus can comprise a plurality of reaction chambers separated from a plurality of trapping chambers by a H₂S permeable membrane, with the reaction chambers and trapping chambers each having buffer component(s) and/or reactive agents that expose the incoming sample to a particular pH and chemical environment in order to allow for the selective liberation and trapping of hydrogen sulfide from the sample.

In a preferred embodiment, the H₂S detecting apparatus features three reaction chambers, namely: a free sulfide reaction chamber having a pH from about 7.0 to about 7.5; an acid labile sulfide reaction chamber having a pH from about 2.6 to about 6.0; and a total sulfide reaction chamber having a pH from about 2.6 to about 6.0 and a reducing agent. Three corresponding trapping chambers can be positioned adjacent to the plurality of reaction chambers such that H₂S gas released from the reaction chambers will diffuse across the H₂S-permeable membrane and into the corresponding trapping chamber. The trapping chambers each have an alkaline environment with a pH from about 9.5 to about 10.0 in order to re-dissolve and trap the hydrogen sulfide gas. Detection can then be accomplished by one of the three following methods: (a) electrochemical, (b) fluorescence, or (c) colorimetric.

The H₂S detecting apparatus of one embodiment of the present invention can also feature a cap, an injection chamber, and a base. The cap can be positioned adjacent to the injection chamber to allow a test sample to be injected through the cap and into the injection chamber. The injection chambers can be in fluid communication with the reaction chambers via a plurality of inlets. The base can be positioned adjacent to the plurality of trapping chambers. The base can be transparent to enable fluorimetric or colorimetric detection of H₂S in the adjacent trapping chambers. The base can also feature a plurality of electrode systems to enable electrochemical detection of H₂S in the adjacent trapping chambers.

The H₂S detecting apparatus of one embodiment of the present invention enables the simultaneously detection of free H₂S, acid labile amounts of H₂S, bound sulfane sulfur available H₂S, and overall total bioavailable H₂S from a single test sample. The concentration of H₂S in the various pools can be calculated as follows: the free H₂S and total H₂S concentrations will be equal to the detected concentrations in the free sulfide trapping chamber and total sulfide trapping chamber, respectively. The acid labile H₂S amount can be determined by subtracting the amount measured in the free sulfide trapping chamber from that of the acid labile sulfide trapping chamber. Lastly, the bound H₂S concentration can be determined by subtracting the acid labile trapping chamber concentration from the total sulfide trapping chamber concentration.

The presently claimed invention is related to methods and hydrogen sulfide (H₂S) detecting apparatuses comprising a single reaction chamber defining a first volume, a single trapping chamber positioned adjacent to the reaction chamber defining a second volume, and an H₂S-permeable membrane positioned between and separating the reaction chamber and the of trapping chamber, wherein the first volume is greater than the second volume. According to a further embodiment the first volume is between 4 and 7 times as large as the second volume. According to a further embodiment the reaction chamber is substantially defined by an interior of walls of a base and the membrane the trapping chamber is defined by an interior of walls of a lid and the member. According to a further embodiment further comprising a deposit passage to access and deposit a sample into the reaction chamber, the deposit passage one of extending from the walls of the base and being defined by a bore in the walls of the base. According to a further embodiment the apparatus further comprising a testing passage to access the testing chamber, the testing passage one of extending from the walls of the lid and being defined by a bore in the walls of the lid. According to a further embodiment the apparatus further comprising one of the base and the lid, the lid and the membrane, and the base, the lid, and the membrane being opaque. According to a further embodiment the apparatus further comprising the lid and the base being hermetically sealed to one another. According to a further embodiment the lid and the base are sonically sealed to one another. According to a further embodiment the apparatus further comprising one or more feet extending from the base to stabilize the apparatus. According to a further embodiment the apparatus further comprising one or more feet extending from the base to stabilize the apparatus wherein the feet are oriented on an opposite side of the apparatus from the deposit passage. According to a further embodiment the apparatus further comprising one of a fluid tight deposit cap removably located in and sealing off a deposit passage, a fluid tight testing cap removably located in and sealing off a testing passage, and both a testing cap and a deposit cap. According to a further embodiment the reaction chamber is preloaded with a buffer to make the reaction chamber environment acidic, with a pH below 6. According to a further embodiment the trapping chamber is preloaded with a buffer to make the trapping chamber environment basic, with a pH above 8. According to a further embodiment an inner wall of the lid is concave and forms a conical recess into the inner wall of the lid, and the tip of the conical recess is circumferentially aligned with a center of the H₂S permeable membrane. According to a further embodiment a fluorescent chemical that binds to HS⁻ is preloaded into the trapping chamber. According to a further embodiment the membrane is permeable to H₂S, but substantially impermeable to HS−. According to a further embodiment the trapping chamber contains a pH above 9 of a Tris base buffer containing one of 0.1 mM DPTA (Diethylenetriaminepentaacetic acid) and MBB (monobromobimane), and the reaction chamber contains a pH below 3 of a phosphate buffer containing 0.1 mM DPTA. According to a further embodiment the reaction chamber further contains 1 mM TCEP (Tris (2-carboxyethyl) phosphine hydrochloride). According to a further embodiment the lid defines an elevated spacing and a deposit passage extends substantially orthogonally to a plan defined by the membrane.

The presently claimed invention is further related to methods and hydrogen sulfide (H₂S) detecting apparatuses comprising a single reaction chamber defining a first volume, a single trapping chamber positioned adjacent to the reaction chamber defining a second volume, an H₂S-permeable membrane positioned between and separating the reaction chamber and the of trapping chamber, the reaction chamber being substantially defined by an interior of walls of a base and the membrane, the trapping chamber being defined by an interior of walls of a lid and the member, a testing passage to access the testing chamber, the testing passage one of extending from the walls of the lid and being defined by a bore in the walls of the lid, one of the base and the lid, the lid and the membrane, and the base, the lid, and the membrane being opaque, the lid and the base being sonically welded and hermetically sealed to one another, one or more feet extending from the base to stabilize the apparatus, wherein the first volume is greater than the second volume, the first volume being between 5 and 6 times as large as the second volume, trapping chamber contains a pH above 9 of a Tris base buffer containing one of 0.1 mM DPTA (Diethylenetriaminepentaacetic acid) and MBB (monobromobimane), and the reaction chamber contains a pH below 3 of a phosphate buffer containing 0.1 mM DPTA.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 illustrates the three biological pools of sulfide found in organisms.

FIG. 2 is an exploded view of an embodiment of a hydrogen sulfide detecting apparatus exemplifying the principles of one embodiment of the present invention.

FIG. 3 is a top view of an embodiment of the reaction chamber of the hydrogen sulfide detecting apparatus depicted in FIG. 2.

FIG. 4 illustrates representative reaction chamber conditions and the electrochemical detection methodology employed by a hydrogen sulfide detecting apparatus exemplifying the principles of one embodiment of the present invention.

FIG. 5 illustrates the diffusion of hydrogen sulfide across a polydimethyl-siloxane (PDMS) membrane of different thicknesses, showing the transfer efficiency of hydrogen sulfide gas transfer from the reaction chambers to the trapping chambers of the hydrogen sulfide detecting apparatus depicted in FIG. 2. The transfer efficiency was measured with high-performance liquid chromatography (HPLC) in conjunction with fluorimetric based methods using monobromobimane (MBB) to derivatize free H₂S.

FIG. 6 illustrates an embodiment of a manufacturing process for the creation of a hydrogen sulfide detecting apparatus exemplifying the principles of one embodiment of the present invention.

FIG. 7 is an exploded view of an alternative embodiment of a hydrogen sulfide detecting apparatus exemplifying the principles of one embodiment of the present invention.

FIG. 8 is a front view of a hydrogen sulfide detecting apparatus according to a further embodiment of the present invention;

FIG. 9 is a sectional view of the hydrogen sulfide detecting apparatus shown in FIG. 8 along the sectional line F8;

FIG. 10 is a top view of the hydrogen sulfide detecting apparatus shown in FIG. 8;

FIG. 11 is an isometric view of the hydrogen sulfide detecting apparatus shown in FIG. 8;

FIG. 12 is an exploded view of the hydrogen sulfide detecting apparatus shown in FIG. 8;

FIG. 13 is a back view of the hydrogen sulfide detecting apparatus shown in FIG. 8;

FIG. 14 is a bottom view of the hydrogen sulfide detecting apparatus shown in FIG. 8;

FIG. 15 is a side view of the hydrogen sulfide detecting apparatus shown in FIG. 8;

FIG. 16 is a front view of a hydrogen sulfide detecting apparatus according to a yet further embodiment of the present invention;

FIG. 17 is a sectional view of the hydrogen sulfide detecting apparatus shown in FIG. 16 along the sectional line F17;

FIG. 18 is a top view of the hydrogen sulfide detecting apparatus shown in FIG. 16;

FIG. 19 is an isometric view of the hydrogen sulfide detecting apparatus shown in FIG. 16;

FIG. 20 is an exploded view of the hydrogen sulfide detecting apparatus shown in FIG. 16;

FIG. 21 is a back view of the hydrogen sulfide detecting apparatus shown in FIG. 16;

FIG. 22 is a bottom view of the hydrogen sulfide detecting apparatus shown in FIG. 16; and

FIG. 23 is a side view of the hydrogen sulfide detecting apparatus shown in FIG. 16.

### DETAILED DESCRIPTION OF THE PREFERRED EMBODIMENTS

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)−(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

Detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Alternate embodiments may be devised without departing from the spirit or the scope of the invention. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward.

As used herein, the terms “a” or “an” are defined as one or more than one. The term “plurality,” as used herein, is defined as two or more than two. The term “another,” as used herein, is defined as at least a second or more. The terms “comprises,” “comprising,” or any other variation thereof are intended to cover a non-exclusive inclusion, such that a process, method, article, or apparatus that comprises a list of elements does not include only those elements, but may include other elements not expressly listed or inherent to such process, method, article, or apparatus. An element proceeded by “comprises . . . a” does not, without more constraints, preclude the existence of additional identical elements in the process, method, article, or apparatus that comprises the element. The terms “including,” “having,” or “featuring,” as used herein, are defined as comprising (i.e., open language). The term “coupled,” as used herein, is defined as connected, although not necessarily directly, and not necessarily mechanically. As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. Relational terms such as first and second, top and bottom, right and left, and the like may be used solely to distinguish one entity or action from another entity or action without necessarily requiring or implying any actual such relationship or order between such entities or actions.

As used herein, the abbreviation CA refers to chronoamperometry; the abbreviation DPV refers to differential pulse voltammetry; the abbreviation DTPA refers to diethylenetriamine pentaacetate; the abbreviation HPLC refers to high-performance liquid chromatography; the abbreviation PCR refers to polymerase chain reaction; and the abbreviation PDMS refers to polydimethyl-siloxane.

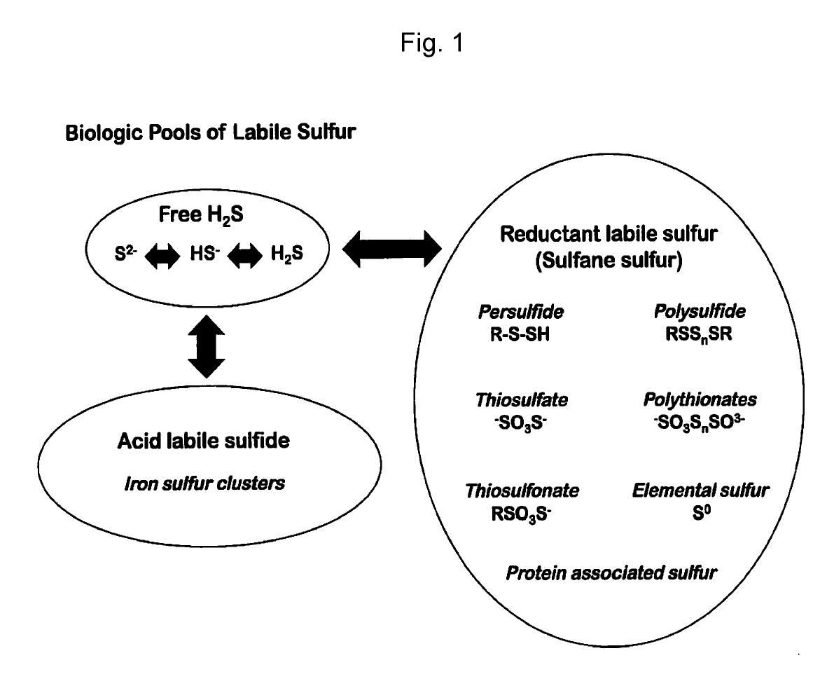

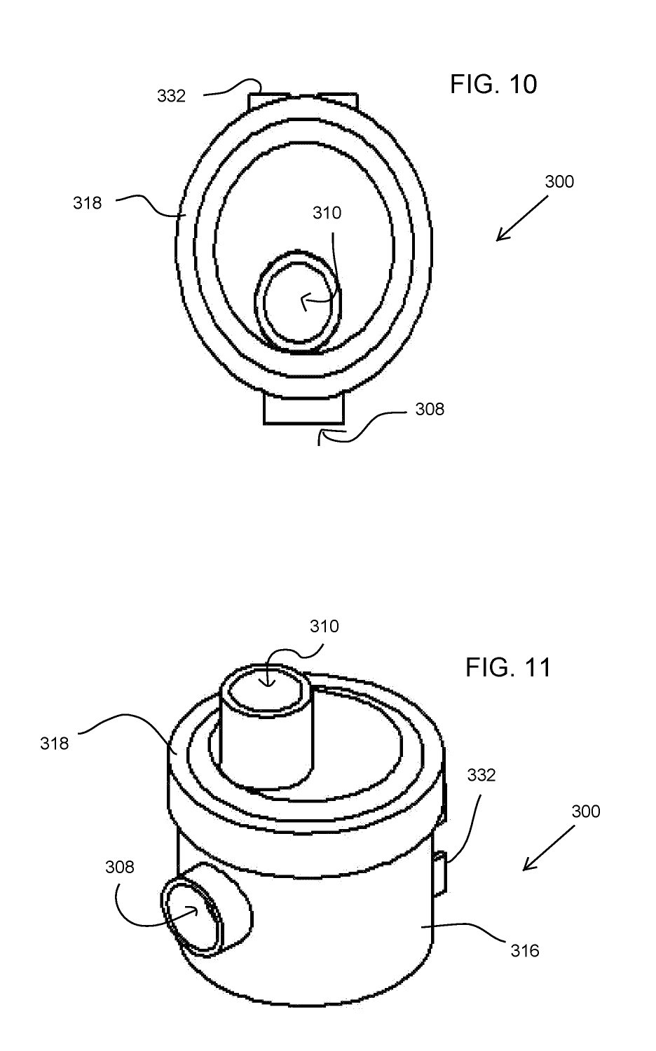

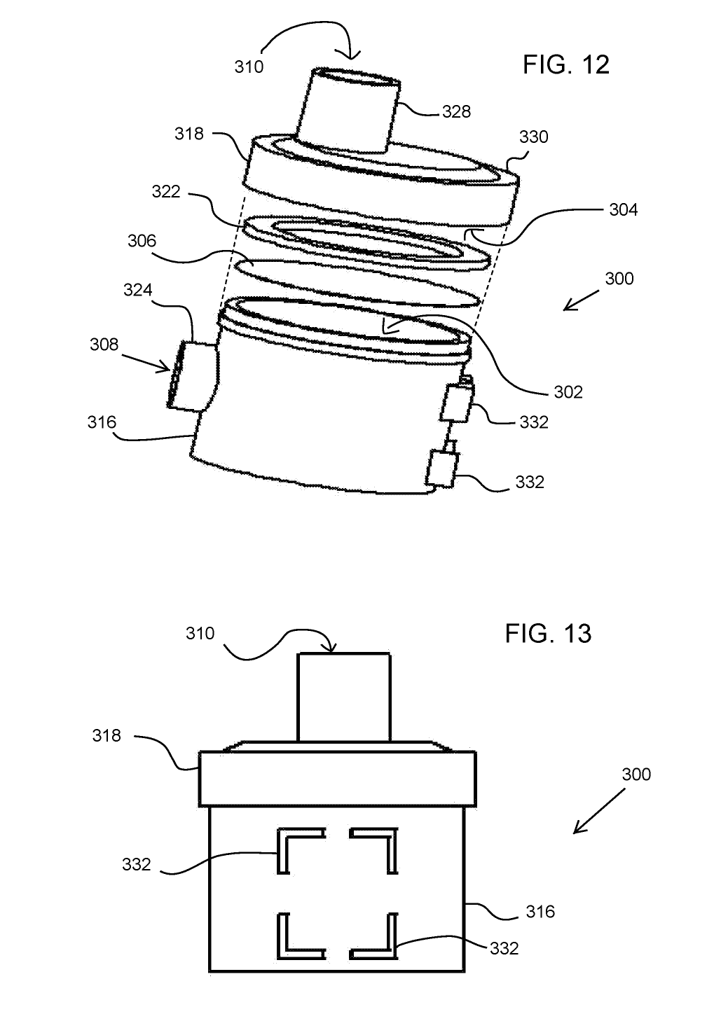

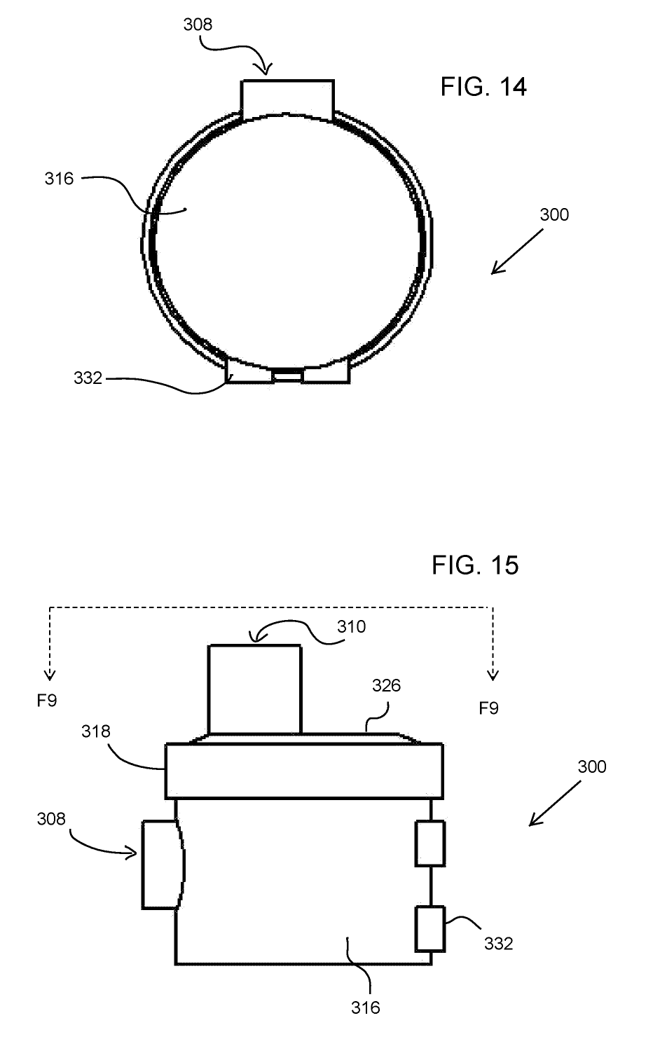

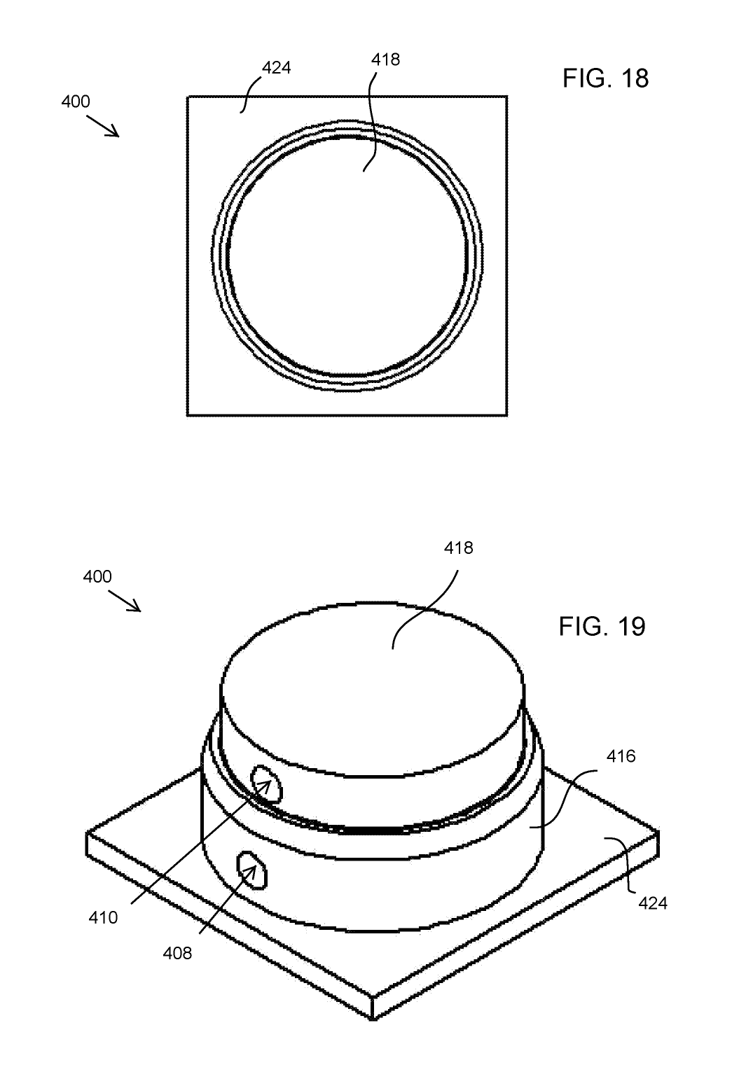

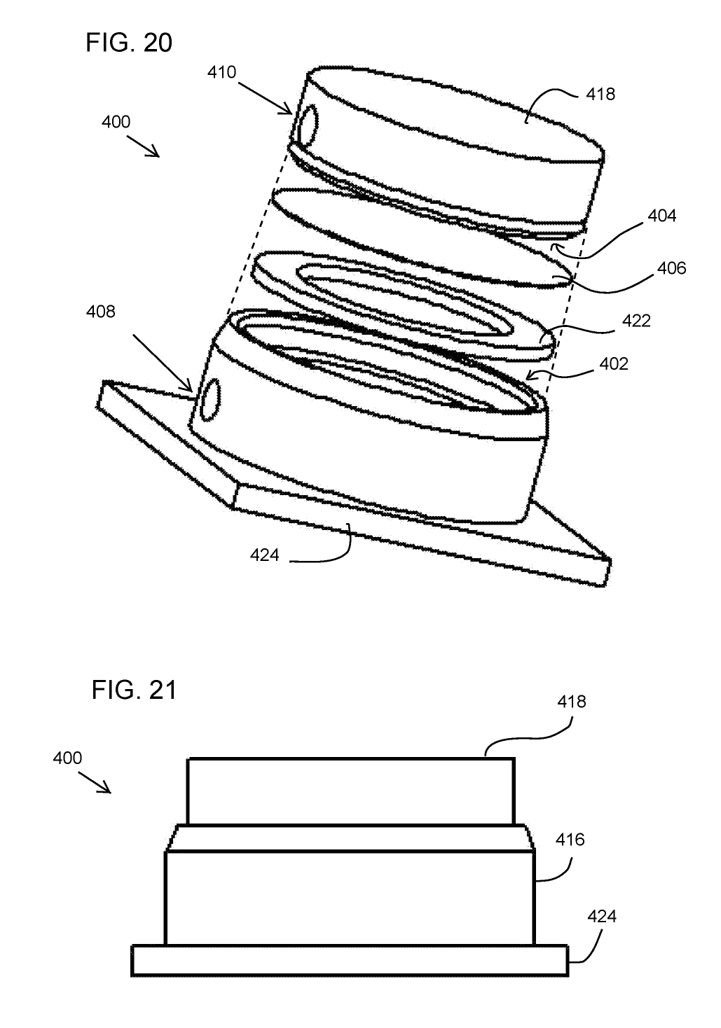

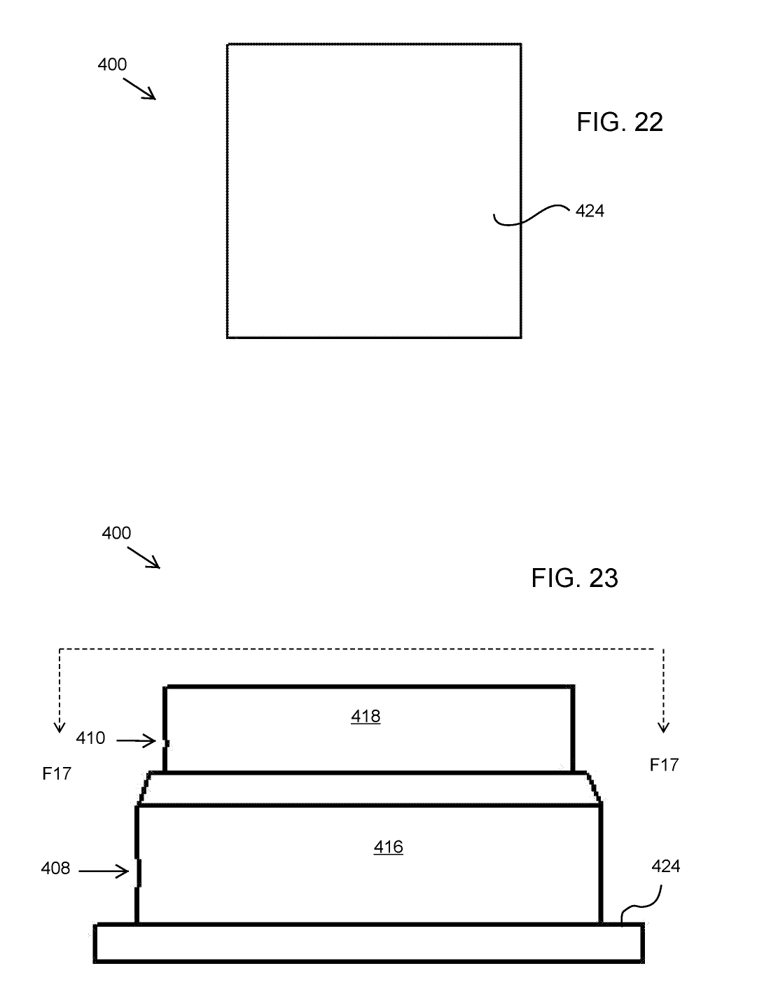

Turning to FIGS. 1-23, various embodiments of the present invention are described. To avoid redundancy, repetitive description of similar features may not be made in some circumstances.

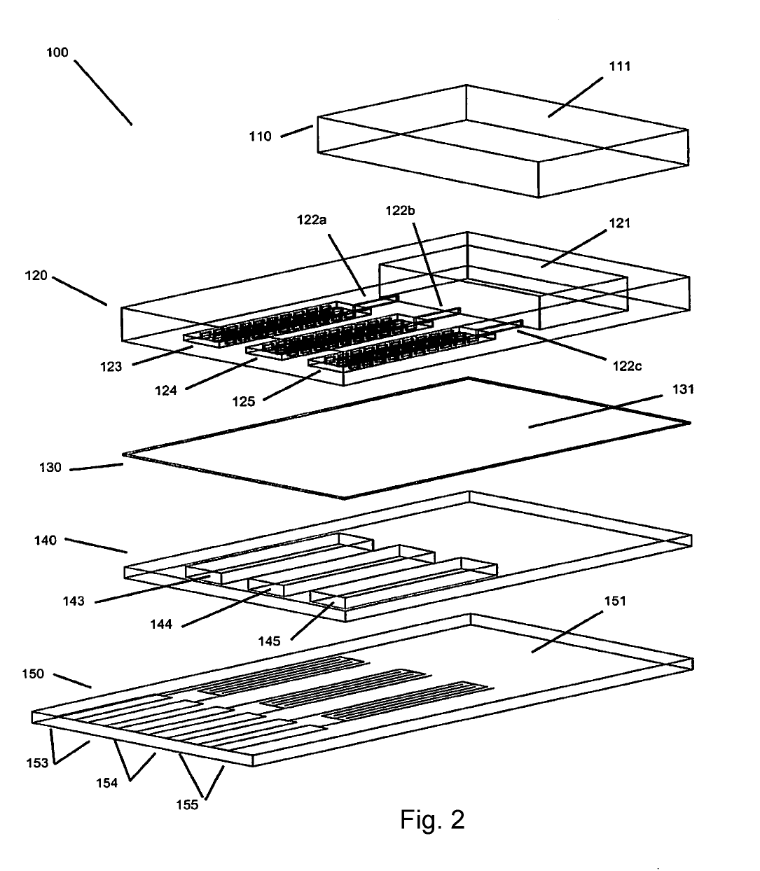

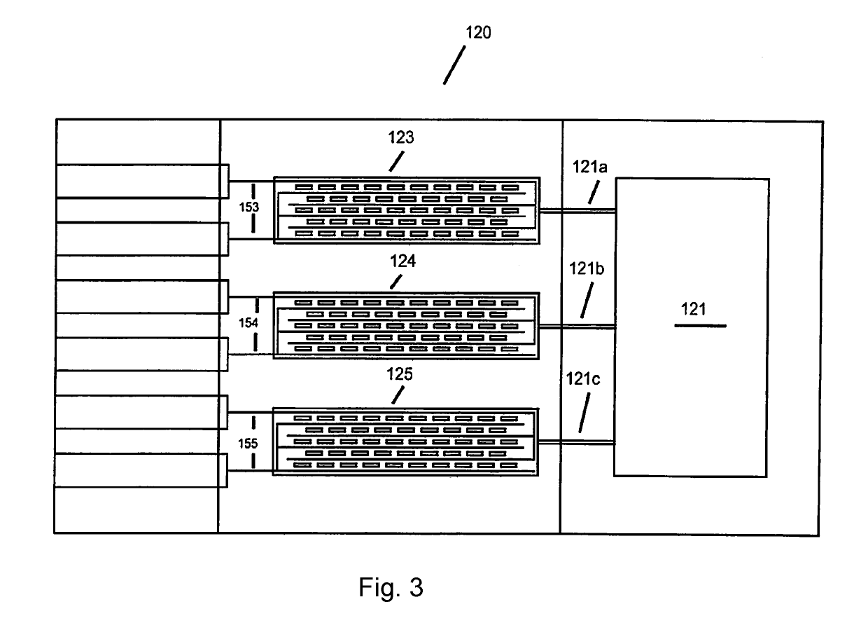

An embodiment of a hydrogen sulfide detecting apparatus exemplifying the principles of the embodiment of the present invention is shown in FIGS. 2 and 3. The apparatus 100 is a lab-on-a-chip device that allows H₂S in its various bioavailable forms to be measured in a reliable, analytical fashion employing unique reaction chemistry, micro-manufacturing techniques, and selective electrochemical measurement techniques. In particular, the apparatus 100 allows for rapid measurement of H₂S from the free H₂S, acid labile sulfide, and sulfane sulfur (polysulfide/persulfide) pools by featuring at least three reaction chambers each having a particular pH and reaction chemicals present to allow for the selective liberation and trapping of H₂S from these pools. The apparatus 100 can be used for research, environmental, and clinical diagnostic purposes in determining hydrogen sulfide bioavailability in biological or other samples. The apparatus 100 is particularly useful for the detection of H₂S in plasma or any other biological or liquid sample. In a preferred embodiment, the apparatus 100 is designed for a single use; however, the apparatus 100 could be configured to be reusable in other embodiments.

Still referring to FIGS. 2 and 3, an embodiment of the hydrogen sulfide detecting apparatus 100 can comprise: a cap 111; an injection chamber 121; a plurality of reaction chambers 123, 124, 125; a permeable membrane 131; a plurality of trapping chambers 143, 144, 145; and a base 151. The cap 111 is preferably constructed out of butyl rubber and is positioned adjacent to the injection chamber 121 to allow a test sample to be injected through the cap 111 and into the injection chamber 121. The injection chamber 121 is in fluid communication with the reaction chambers 123, 124, 125 via inlet channels 122 a-c. The permeable membrane 131 separates the reaction chambers 123, 124, 125 from adjacent trapping chambers 143, 144, 145. The trapping chambers 143, 144, 145 are positioned adjacent to electrode systems 153, 154, 155 on the base 151.

In a preferred embodiment, the injection chamber 121 comprises a single piece PDMS-molded (polydimethyl-siloxane) chamber which is evacuated of air and adapted to receive fluid injected directly into it. A first inlet channel 122 a connects the injection chamber 121 to free sulfide reaction chamber 123; a second inlet channel 122 b connects the injection chamber 121 to the acid labile sulfide reaction chamber 124; and a third inlet channel 122 c connects the injection chamber 121 to the total sulfide-reaction chamber 125. In this arrangement, the reaction chambers 123, 124, 125 are reproducibly filled with uniform volumes from a single injection while minimizing diffusion of buffer components and reaction products between the chambers.

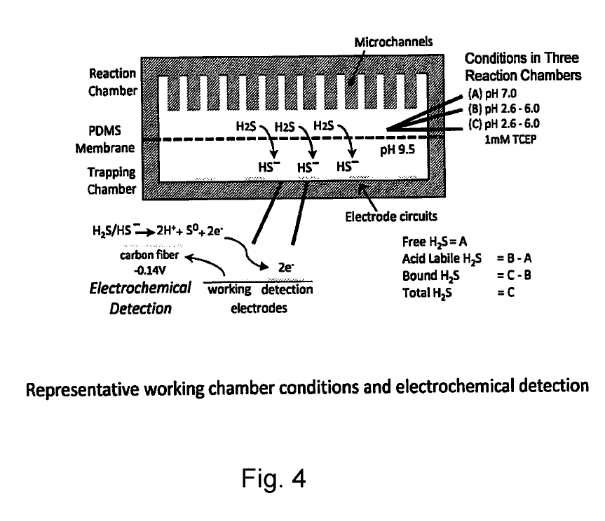

Each reaction chamber 123, 124, and 125 preferably comprises interdigitated microchannels of PDMS with dried or powder-coated buffer components and/or reactive agents that expose the incoming sample to a particular pH and chemical environment in order to allow for the selective liberation and trapping of hydrogen sulfide. FIG. 4 illustrates representative reaction chamber conditions of the hydrogen sulfide detecting apparatus 100. Free or volatilized hydrogen sulfide is derivatized in alkaline conditions, preferably pH>7.0. The derivatization preferably occurs under low oxygen conditions, preferably <5% oxygen, more preferably <2% oxygen, and most preferably <1% oxygen. Accordingly, in a preferred embodiment, the free sulfide-reaction chamber 123 is at neutral pH environment (pH from about 7.0 to about 7.5). Meanwhile, the release of hydrogen sulfide from the acid labile pool generally requires a pH less than 5.4. Thus the determination of acid labile sulfide involves acidification of the sample, preferably pH<4.0, more preferably from about pH 2.0 to about pH 3.0, and most preferably about pH 2.6, thereby causing release of free hydrogen sulfide from the acid labile pool. Accordingly, the acid labile sulfide-reaction chamber 124 is preferably at an acid pH environment (pH from about 2.6 to about 6.0). Lastly, the total sulfide-reaction chamber 125 is preferably at an acidic pH environment (pH from about 2.6 to about 6.0) with a reducing agent present such as 1 mM tris (2-carboxyethyl) phosphine hydrochloride (TCEP). The total labile sulfide amount, including the sulfane sulfur component along with the acid-labile and free sulfide, is determined by using a reducing agent with an acid solution. The reducing agent is preferably TCEP, which cleaves disulfide bonds to liberate the sulfane sulfur atom. While dithiothreitol (DTT) could also be used, TCEP is preferred because it is water soluble, non-volatile, reduces disulfide bonds more rapidly and has been shown to be very stable across a wider range of pH (2.0-9.5) than DTT. TCEP does not have a thiol moiety and has the additional advantage of not requiring thiol removal prior to reaction with MBB. By contrast DTT contains a thiol moiety and has been reported to have small amounts of sulfide contaminants.

The permeable membrane 131 is positioned between the reaction chambers 123, 124, 125 and the corresponding trapping chambers 143, 144, 145. The H₂S permeable membrane 131 is preferably silicone-based or may comprise blended materials such as silicone-polycarbonate blends. The thickness of the H₂S permeable membrane 131 may vary between about 75 μm to about 500 μm or greater depending on device construction, application, and required mechanical strength. Other constructions may utilize membrane materials that include silicone and additive compounds for increased specificity of hydrogen-sulfide permeability. These include, but are not limited to, the combination of silicone and polycarbonate for membranes or dimethyl silicone. Other membrane base materials may be utilized which include but are not limited to composite membranes with silicone or PDMS coating on micro-porous cellulose structure. Membrane fabrication may be completed via microfabrication or other techniques. Preferential techniques include spinning membrane polymer in liquid form onto a flat surface like a silicon-nitride wafer. The membrane may be subsequently released following curing, the removal of entrapped air bubbles and solidification. Other techniques include but are not limited to Reactive Ion Etch (RIE) processes. This includes the deposition of the liquid polymer membrane atop a wafer, and then patterning and removing the wafer substrate to release the membrane for use.

The trapping chambers 143, 144, 145 are positioned beneath the reaction chambers 123, 124, 125 such that H₂S gas released from each reaction chamber will diffuse across the permeable membrane 131 and into the corresponding trapping chamber. All three trapping chambers 143, 144, 145 are filled with an alkaline solution (100 mM NaOH, pH from about 9.5 to about 10) to trap and re-dissolve the hydrogen sulfide gas which diffuses across the permeable membrane 131. In a preferred embodiment, the trapping chambers are constructed out of PDMS. However, other materials and construction processes may be utilized, including but not limited to solid casting, RIE patterning of silicon, and 3-D printing of non-porous chambers using 3-D printing material.

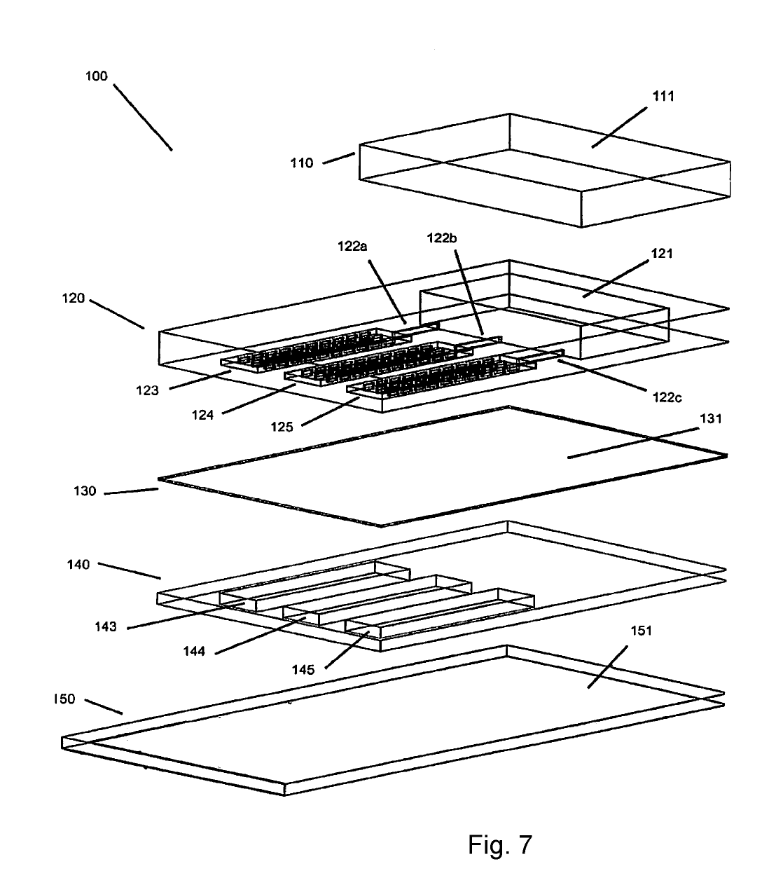

The base 151 is preferably constructed out of plastic. However, it may be constructed out of other base materials including but not limited to silicon, silicon nitride, or metallic materials. In the embodiment depicted in FIGS. 2 and 3, the base 151 features interdigitated electrode systems 153, 154, 155 for electrochemical detection of H₂S in the adjacent trapping chambers 143, 144, 145. The electrode systems 153, 154, 155 can each feature a working electrode, a counter electrode, and a reference electrode. The working electrodes can be constructed out of inert metals such as gold, silver or platinum, are preferably medical grade carbon fibers (6 μm diameter; 12 Ω-cm). Other embodiments may include use of graphene or similar thin-sheet materials for electrode application. The working electrodes can be fixed in the base 151 longitudinally so as to allow exposure to the sample in the trapping chambers 143, 144, 145. The counter electrode is preferably constructed out of an electrochemically inert material such as gold, platinum, or carbon and can be fixed in the base 151 parallel to the working electrode (preferably with 1 mm-5 mm separation) in order to detect electrons produced by the oxidation reaction occurring in the trapping chambers 143, 144, 145. The reference electrode is preferably constructed out of platinum or Ag/AgCl wire in electrical contact with the specimen. Alternatively, and as depicted in FIG. 7, the base 151 can be constructed out of a transparent material (e.g., plastic) to allow H₂S levels in the trapping chambers 143, 144, 145 to be detected via fluorescence, chemiluminescence and colorimetric detection. In another alternative embodiment, the base 151 can be constructed out of a transparent material while also featuring electrode systems, thereby providing a user the option of detection methods.

In operation, a sample can be deposited into the injection chamber 121 by using a needle to penetrate the cap 111. The sample will be transmitted in uniform volumes to the free sulfide reaction chamber 123, the acid labile sulfide reaction chamber 124, and the total sulfide-reaction chamber 125 via the first inlet channel 122 a, the second inlet channel 122 b, and the third inlet channel 122 c, respectively. The releasing chambers 123, 124, 125 are separated from their corresponding trapping chambers 143, 144, 145 by the H₂S permeable membrane 131. In the free sulfide-reaction chamber 123, only free H₂S gas will diffuse across the membrane 131 into the free sulfide trapping chamber 143. In the acid labile sulfide-reaction chamber 124, both the free H₂S and acid labile H₂S pools will diffuse across the membrane 131 into the acid labile sulfide trapping chamber 144. In the total sulfide-reaction chamber 125, H₂S from all three pools (free, acid labile, and bound sulfane sulfur) are released and will diffuse into the corresponding total sulfide trapping chamber 145. Upon entry into the trapping chambers 143, 144, 145, the H₂S is converted from its gaseous form into the HS″ form due to the presence of basic (pH ⁻9.5-10.0) conditions. The concentration of H₂S in the various pools then can be calculated as follows: the free H₂S and total H₂S concentrations is equal to that measured by the free sulfide trapping chamber 143 and total sulfide trapping chamber 145, respectively. The acid labile H₂S amount is determined by subtracting the amount measured in the free sulfide trapping chamber 143 from that of the acid labile sulfide trapping chamber 144. The bound H₂S concentration is determined by subtracting the acid labile trapping chamber 144 concentration from the total sulfide trapping chamber 145 concentration. In this way, the device simultaneously detects free H₂S, acid labile amounts of H₂S, bound sulfane sulfur available H₂S, and overall total bioavailable H₂S from one specimen.

Electrochemical Detection

As depicted in FIGS. 2 and 4, the hydrogen sulfide detecting apparatus 100 can feature a base 151 having integrated electrode systems 153, 154, 155 for the electrochemical detection of H₂S in the trapping chambers 143, 144, 145. Contemporary electrochemical methods used for in vivo and in vitro detection of biological compounds are chronoamperometry (CA) and differential pulse voltammetry (DPV). Both CA and DPV utilize a three-electrode system (reference, counter and working). In CA, the working electrode potential is held constant (with respect to the reference electrode) and current is measured as a function of time. Excellent temporal resolution and sensitivity is achieved with this technique. However, the origin of the current cannot be discriminated, for the measured current is a superposition of any species that is electrolyzed at or below the working electrode potential. For single species concentration determination, selective working electrodes must be used.

DPV is a hybrid of traditional cyclic voltammetry and CA. The sensitivity is similar to CA, but the temporal resolution is less. DPV has a potential applied to the working electrode that is a linearly increasing pulse train. The difference in current per pulse is recorded as a function of a linearly increasing voltage. Current is measured at two points for each pulse, the first point just before the pulse application and the second at the end of the pulse. This technique yields a curve with a peak that is directly proportional to species concentration. This allows for concentration discrimination of species in solution whose half-wave potential differs by as little as 40 to 50 mV.

H₂S has an oxidation reaction at −0.14 V producing two electrons through HS⁻ reaction with Fe(CN)₆ to yield the overall equation of: H₂S→S+2H++2e⁻. Both CA and DPV can detect the electrons generated from HS⁻ oxidation. And since both modalities are incorporated in contemporary potentiostats, both can be used for determining optimal electrochemical detection. During CA detection, the working electrode is fixed between −0.20-0.30 V to oxidize H₂S/HS⁻, and during DPV a range of voltages is applied. The voltages for electron detection must sweep from −0.3 V to 0.3 V with a scan rate of 5-10 mV/sec and a scan increment 2-4 mV. An example of pulsing parameters are a pulse height of 25 mV, a step/drop time of 100 ms, and a pulse width of 50 ms; although these may vary by 50% depending on chip performance.

FIG. 4 illustrates one embodiment of working chamber conditions and electrochemical detection methodology. Electrochemical detection of H₂S is performed in the three separate trapping chambers: the free sulfide trapping chamber 143; the acid labile sulfide trapping chamber 144; and the total sulfide trapping chamber 145. As explained above, the free sulfide reaction chamber 123 is preferably at neutral pH 7.0, the acid labile sulfide reaction chamber 124 at acid pH (pH from about 2.6 to about 6), and the total sulfide reaction chamber 125 at acid pH (pH from about 2.6 to about 6) plus 1 mM TCEP to liberate bound H₂S. Hydrogen sulfide gas will diffuse across the permeable membrane and be trapped in the corresponding trapping chambers due to the basic conditions present (pH from about 9.5 to about 10.0). FeCN will oxidize HS− to produce 2e−. The changes in electrochemical potential can be measured using a potentiostat coupled to the embedded electrodes. The potentiostat can be coupled to the integrated electrode systems 153, 154, 155, preferably via a copper wire that is adhered to the electrodes with a silver epoxy.

Fluorescence, Chemiluminescence and Colorimetric Detection

As depicted in FIG. 7, the hydrogen sulfide detecting apparatus 100 can alternatively feature a transparent base 151 to allow H₂S levels in the trapping chambers 143, 144, 145 to be detected via fluorescence, chemiluminescence and colorimetric dyes. Dyes such as bimane compounds, including but not limited to dibromobimane, monobromobimane, benzodithiolone, and dansyl azide, can be used in conjunction with fluorescence excitation and emission spectrometry to detect sulphide. Hydrogen sulphide reaction with electron-poor aromatic or other electrophilic chemicals can produce color shifts in the visible light spectrum. For example nitrobenzofurazan thioether compounds can react to form nitrobenzofurazan thiol with a shift in absorbance spectrum at 534 nm. Hydrogen sulphide contained within the trapping chambers 143, 144, 145 can be detected by chemiluminescence through reaction with ozone or other electrophilic compounds to stimulate photon release.

Device Fabrication

A hydrogen sulfide detecting apparatus exemplifying the principles of one embodiment of the present invention can be fabricated utilizing a variety of materials and techniques. One preferred method is to fabricate in layers via PDMS. Alternate polymer materials, apart from PDMS may be utilized that include SU-8 polymers or similar structures. Additives to the base material may be employed, such as polyethylene oxide (PEO). These additives can increase the capillary action of the devices. Other methods include but are not limited to the use of silicon or metals such as copper. For example, a suitable microfabrication procedure would be to utilize bulk micro-machined silicon wafers that serve as the device substrate. Alternate fabrication processes may be utilized including layer-by-layer deposition through advanced printing and processing, but not limited to 3D printing. Casting via mold-and-pour could also be used to generate the appropriate structures given non-permeable materials.

In a preferred embodiment, the hydrogen sulfide detecting apparatus of one embodiment of the present invention is constructed in layers utilizing PDMS construction in combination with other polymer materials. For example, the hydrogen sulfide detecting apparatus 100 depicted in FIG. 2 is comprised of five layers. Referring to FIG. 2, the first layer 110 can comprise a butyl rubber cap 111. The second layer 120, which is bonded to the first layer 110, can be cast from a reusable mold to form the injection chamber 121, the plurality of reaction chambers 123, 124, 125, and inlet channels 122 a-c. The third layer 130 can comprise a PDMS membrane 131 and is bonded to the second layer 120. The thickness of the membrane 131 may be varied depending on the material used and fabrication technology employed. The fourth layer 140 is bonded to the third layer 130 and comprises a plurality of trapping chambers 143, 144, 145 filled with a trapping buffer (e.g., 100 mM NaOH, pH from about 9.5 to about 10). In the preferred construction, the fourth layer 140 is a 1 mm thick section of PDMS that has trapping chambers 143, 144, 145 cut out of the PDMS material and aligned with the reaction chambers 123, 124, 125. The fifth layer 150 preferably consists of a plastic base 151 with interdigitated electrode systems 153, 154, 155 for electrochemical detection of the test specimen. The electrode systems 153, 154, 155 can be printed on the surface via microfabrication techniques and aligned with the trapping chambers 143, 144, 145 formed by cutouts in the fourth layer 140. The fifth layer 150 preferably is longer than the fourth layer 140, allowing access to the electrodes on the apparatus 100. For example, the first layer 110 can be 10 nm×2 nm, the second, third, and fourth layers 120, 130, 140 can be 40 nm×2.5 nm, and the fifth layer 150 can be 50 nm×2.5 nm. Other embodiments of the chip design could feature either increased or reduced dimensions to enable detection of larger or smaller volumes, respectfully. Finally, the fifth layer 150 is bonded to the fourth layer 140 and air is evacuated from the injection and releasing chambers.

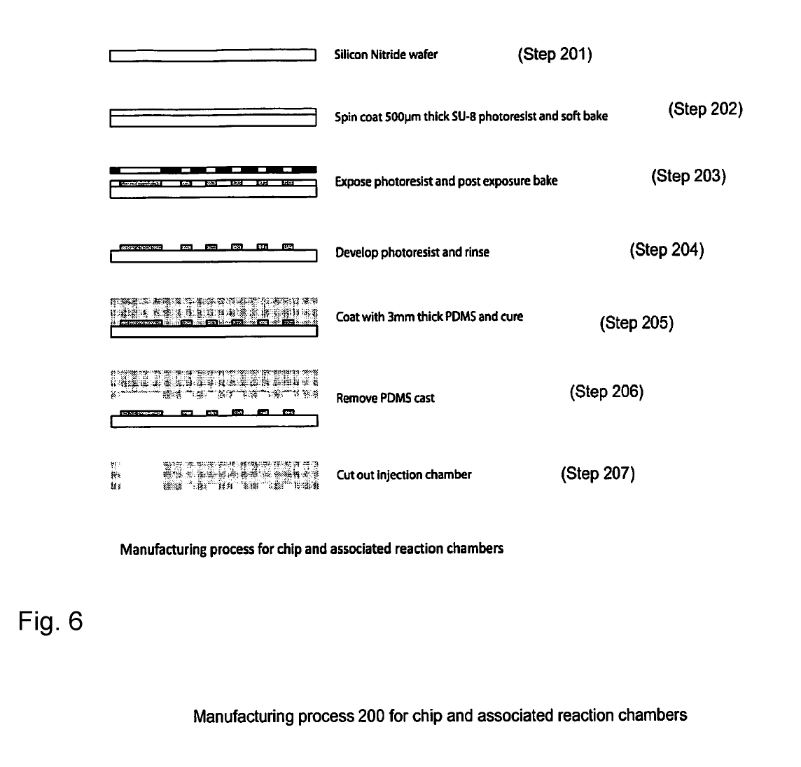

FIG. 6 illustrates an exemplary process 200 for manufacturing the second layer 120 of the hydrogen sulfide detecting apparatus 100. In the preferred PDMS fabrication, a silicon nitride wafer is provided in step 201. In step 202, the silicon nitride wafer is spin-coated with 500 μm thick SU-8 photoresist and soft baked. Next, the wafer is exposed through a lithographic mask and baked post exposure (step 203). The photoresist is then developed and rinsed in step 204. In step 205, uncured PDMS is poured onto the mold and cured. In the preferred PDMS polymer construction, the cast is removed in step 206 and the injection chamber is cut out through the entire thickness of the cast in step 207. The required chemical reaction buffers for the acid labile (acid pH from about 2.6 to about 6.0) and total sulfide (acid pH plus 1 mM TCEP) can be coated by evaporation of concentrated solutions on to the surface of the respective chambers 124, 125 to complete the second layer 120. Final height of PDMS material should be high enough to encapsulate the designed channels with heights to 2000 microns. Design features include high surface areas consisting of, but not limited to, capillary channels that range from 1 to 400 microns in width with heights variable from 10 to 2000 microns. However, one skilled in the art will appreciate that channel height is determined based on required sample volumetric size. Additionally, one skilled in the art will recognize that the foregoing process may also be utilized for manufacturing the fourth layer 140 of the hydrogen sulfide detecting apparatus 100.

### EXAMPLES

### Example 1

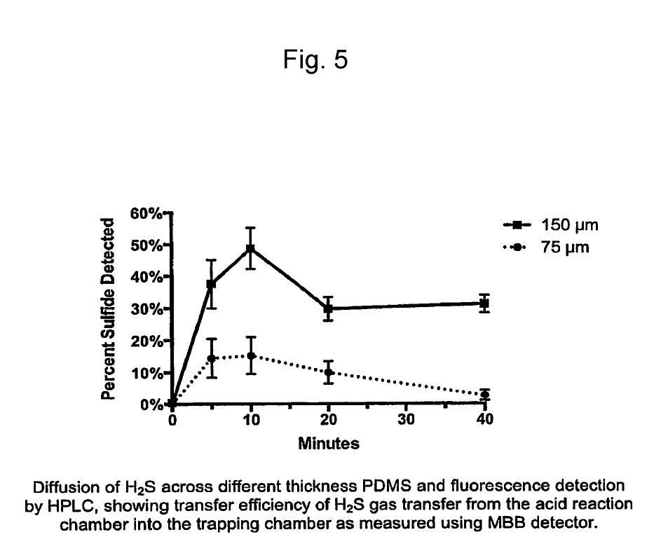

The transfer efficiency of H₂S across 75 μm and 150 μm PDMS membranes was demonstrated using an embodiment of a hydrogen sulfide detecting apparatus exemplifying the principles of one embodiment of the present invention. A sample was introduced into a single acid reaction chamber separated by a 75 μm PDMS membrane from an alkaline trapping chamber containing 10 mM monobromobimane (MBB). This experiment was repeated with a 150 μm PDMS membrane. The H₂S transfer efficiency over time was measured by RP-HPLC detection of sulfide dibromane (SDB). FIG. 5 illustrates the diffusion of H₂S across a PDMS membrane of different thicknesses, utilizing fluorescent detection by HPLC. The transfer efficiency of H₂S from the acid reaction chamber into the trapping chamber is depicted as measured using a MBB detector for both a 75-μm membrane and a 150-μm membrane. The sodium sulfide volatilized the sulfide anion into H₂S gas, which diffused across the membrane and was trapped in a separate chamber at pH 9.5 with 0.1 mM DTPA. Sample aliquots were taken from the trapping chamber at specified times. The amount of sulfide was detected using fluorescent HPLC analysis as described in PCT/US2013/031354, which is incorporated herein by reference. As shown in FIG. 5, an approximate 15% transfer efficiency occurred within 10 minutes using the 75-μm membrane, while an approximate 50% transfer efficiency occurred within 10 minutes using the 150-μm membrane. The transfer efficiency of H₂S across the permeable membrane can be utilized to calibrate the hydrogen sulfide detecting apparatus 100.

### Example 2

An embodiment of a hydrogen sulfide detecting apparatus exemplifying the principles of one embodiment of the present invention can be used to determine the concentration of H₂S in a specimen using electrochemical, fluorescence, or colorimetric detection methods. In such instances, a blood sample will be obtained from a subject and placed into vacutainer tubes containing lithium heparin (BD Biosciences, Cat. No. 367886), which is then immediately centrifuged at 4° C. at 1500 RCF for 4 minutes to separate the plasma from the red blood cells. The plasma sample will then be injected into the injection chamber 121 of the apparatus 100 via a 26-gauge needle and 1 cc syringe. The sample will be pulled into the injection chamber 121 which is evacuated of air by wicking action, where it will be further pulled into the three parallel reaction chambers 123, 124, 125 for free sulfide, acid labile+free sulfide, and total sulfide detection respectively. The buffer components that coat the chambers will dissolve in the plasma sample, providing the correct pH and chemical concentrations necessary for the reactions to occur at room temperature. After approximately 15 minutes, hydrogen sulfide will be liberated from each of the reaction chambers 123, 124, 125; will diffuse across the membrane 130; and will be trapped in the alkaline buffer in the respective trapping chambers 143, 144, 145. Detection can then be accomplished by one of the three following methods: (a) electrochemical, (b) fluorescence, or (c) colorimetric.

If the electrochemical method is to be employed, the apparatus 100 will be connected to a potentiostat such as the VersaStat 4 (Princeton Applied Research), with one lead each for the working electrode, counter electrode, and reference electrode. A method such as differential pulse voltammetry (DPV) will be used to acquire a signal that is a measure of hydrogen sulfide concentration in the plasma sample. Typical settings for the DPV parameters are 25 mV for pulse height, 50 msec for pulse width, 1 mV for step height, and 100 msec for step width. Peak currents will be obtained for each chamber and converted into sulfide concentrations based on a calibration function (See Example 1).

If a fluorescence method is to be employed, the apparatus 100 will have a fluorescent dye such as dibromobimane (DBB) dissolved in solution in the trapping chambers 143, 144, 145. After reaction between dye and hydrogen sulfide in the trapping chambers 143, 144, 145, fluorescence will be measured using appropriate excitation and emission wavelengths. If DBB dye is used these are 358 nm and 484 nm respectively. Fluorescence will be quantified and converted to sulfide concentrations by means of a calibration function (See Example 1).

If a colorimetric method is to be used, the apparatus 100 will have a compound such as nitrobenzofurazan thioether dissolved in solution in the trapping chambers 143, 144, 145. Upon reaction with sulfide, it will form nitrobenzofurazan thiol, with a shift in the absorbance spectrum at 534 nm as previously noted. Absorbance will be quantified and converted to sulfide concentrations by means of a calibration function (See Example 1).

Free sulfide, acid-labile sulfide, bound sulfane sulfur, and total sulfide can then be calculated as follows. Free sulfide and total sulfide concentrations will be equal to that measured in the free sulfide and total sulfide trapping chambers 143, 145 respectively. The acid labile sulfide concentration will be equal to that measured in the “acid labile+free sulfide” chamber 144 minus the concentration in the free sulfide chamber 143. The bound sulfane sulfur concentration will be found by subtracting the concentration measured in the “acid labile+free sulfide” chamber 144 from that measured in the total sulfide chamber 145.

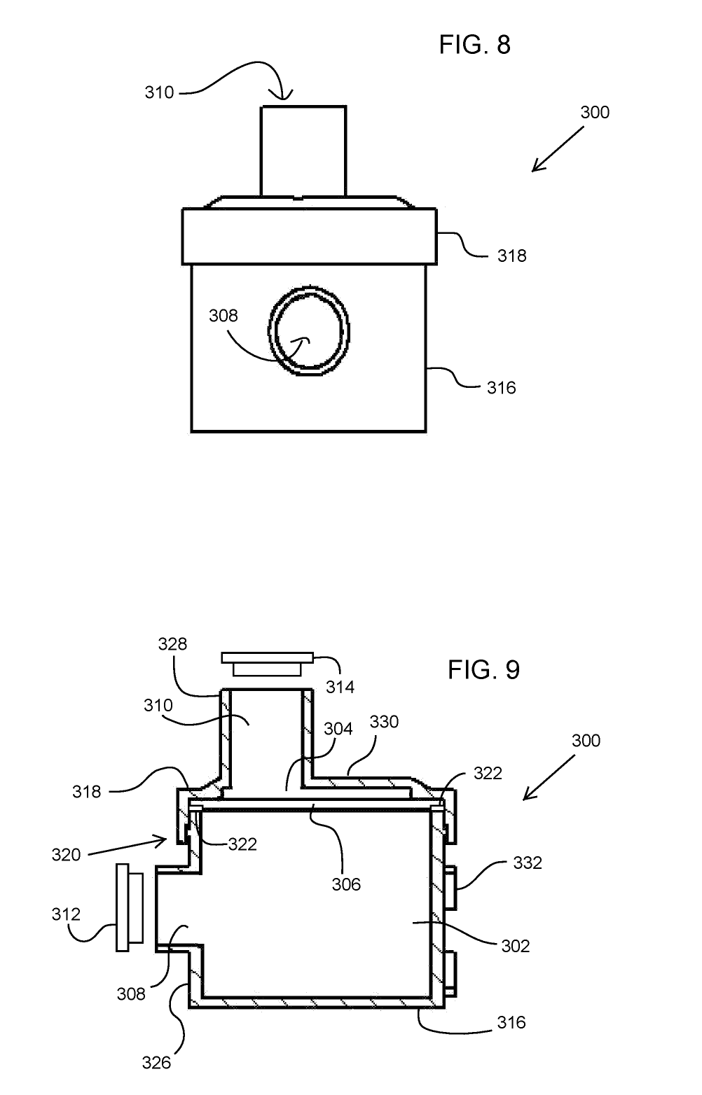

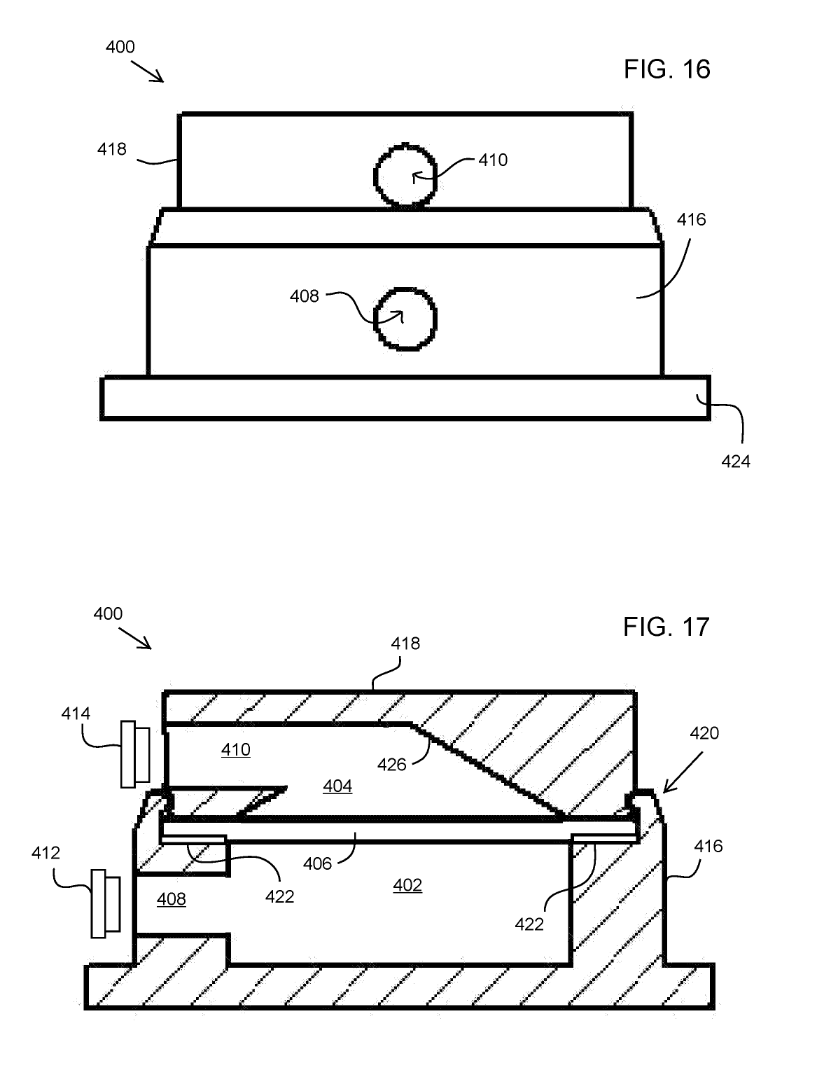

Turning next to FIGS. 8-23 two further embodiments of the hydrogen sulfide detecting apparatus 300, 400 are shown. FIGS. 8-15 show an embodiment preferably for relatively higher volume samples, for example, industrial samples, termed the sinc-1 embodiment. FIGS. 16-23 show an embodiment preferably for relatively lower volume samples, for example, biologic samples, termed the sinc-2 embodiment. Variations of the sinc-1 and sinc-2 embodiments described below are also conceived.

As shown in FIGS. 8-15, the sinc-1 hydrogen sulfide detecting apparatus 300 has a reaction chamber 302 and a trapping chamber 304. The reaction chamber 302 is separated from the trapping chamber 304 by an H₂S permeable membrane 306. A deposit passage 308 to place test material into the reaction chamber 302 extends from the reaction chamber 302. A testing passage 310 to access the trapping chamber 304 extends from the trapping chamber. A fluid tight deposit cap 312 preferably covers the deposit passage 308, and a fluid tight testing cap 314 preferably covers the testing passage 310. The detecting apparatus preferably is formed from a base 316 and a lid 318 hermetically sealed together at a joint 320. One or more gaskets 322 and the membrane 306 are preferably retained between the lid 318 and the base 316 at or adjacent to the joint 320. The deposit passage 308 is defined by a deposit tube 324 extending from a base wall 326. The testing passage 310 is defined by a testing tube 328 extending from a lid wall 330. On an opposite side of the sinc-1 hydrogen sulfide detecting apparatus 300 from the deposit tube 324 is preferably one or more feet 332 formed in the base wall 326. The one or more feet 332 allow for the sinc-1 hydrogen sulfide detecting apparatus 300 to be set securely down with the deposit tube 324 facing upward for easy deposit access of a sample and increased workability. In a preferred embodiment the lid 318 slopes upward creating an elevated spacing 326 in the trapping chamber. The base 316, lid 318, deposit tube 324, and testing tube 328 are preferably formed of an acid and alkaline fast material, such a polypropylene.

Inside the reaction chamber 302 is preferably 20-25 mls of a 2.6 pH phosphate buffer containing 0.1 mM DPTA (Diethylenetriaminepentaacetic acid). Inside the trapping chamber 304 is preferably 4 ml of a 9.6 pH Tris base buffer containing 0.1 mM DPTA. The reaction chamber 302 is preferably between 20 and 30 ml in volume, more preferably 26 ml in volume. The trapping chamber 304 is preferably between 2 and 8 ml in volume, more preferably between 3 and 6 ml, and most preferably 4 ml in volume. The smaller volume of the trapping chamber 304 compared to the reaction chamber 302 allows H₂S to concentrate in lower volume trapping chamber 304.

To use the sinc-1 hydrogen sulfide detecting apparatus 300, a user preferably sets the sinc-1 hydrogen sulfide detecting apparatus 300 on the feet 332 and orients the deposit tube 324 into the upwards direction. The user then removes the deposit cap 312 from the deposit tube 324, places a sample into the reaction chamber 302, and replaces the deposit cap 312. The sample is then allowed to react with the buffer, and H₂S is liberated. The H₂S then migrates across the H₂S permeable membrane from the reaction chamber 302 into the trapping chamber 304. In the alkaline conditions in the trapping chamber, the H₂S loses an H⁺ ion, and becomes HS⁻, a species which no longer freely permeates across the membrane 306. This allows the H₂S to be trapped in the trapping chamber 304 and build up concentration.

After a given amount of time, the testing cap is preferably removed and an electrode 334 is preferably inserted into the testing passage 310 and preferably into the trapping chamber 304. The electrode is allowed to achieve a reading from the H₂S concentration. Then the electrode is preferably removed from the deposit passage 308 and the testing cap 314 is replaced.

The H₂S is trapped by converting H₂S to HS⁻ once it passes through the membrane, and it then prevented from crossing back again. H₂S is converted to HS⁻ in the very alkaline conditions. The conditions may have a pH of above 9, above 11, and above 13. The HS⁻ is preferably read directly with the electrode. Because the HS⁻ anion is preferably the only anion in the trapping chamber, the electrode does not need to measure the HS− directly. Rather the electrode may only measure anion concentration. The electrode preferably looks for peak at 0.05 to 1.05 millivolts, and preferably between 0.45 and 0.55 millivolts. This is a window that tests HS⁻ minus only, not other S compounds. The HS⁻ concentration measured in the trapping chamber is understood to be the free H₂S concentration in the sample.

A current embodiment of the electrode is a flat, elongate gold and platinum screen plated electrode on a hand held potentiaostat. The electrode can be inserted into the trapping chamber 304 through testing passage 310. Alternative embodiments include where the electrode may be smaller and cylindrical. In further embodiments, the electrode may be preloaded into the trapping chamber 304 of the sinc-1 hydrogen sulfide detecting apparatus 300 in a hermetically sealed section. After a given period of time, a barrier between the hermetically sealed section and the portion of the trapping chamber 304 where HS− was building up would be removed, and HS− would be allowed to flow into the formerly hermetically sealed section and read by the electrode. In another further embodiment, the electrode may be preloaded with an H₂S and HS− impermeable film covering the electrode. After the reaction is complete and testing is ready, the film on the electrode is removed and the sample may be tested. In a further embodiment the electrode may be smaller and cylindrical. In further embodiments, the electrode may be formed with gold nanotubes, gold nanowires, heavy metal nanotubes, and polymer coated electrodes such as PDMS (polydimethylsiloxane), for example.

In one embodiment, the DPV technique for electrochemical reading may be used.

The buffer to sample ratio in reaction chamber for the sinc-1 hydrogen sulfide detecting apparatus 300 is preferably at least four times the volume of buffer to volume of sample. Preferably a 0.7 molar solution is used (instead of, for example, 0.07 molar solution as might be used in the sinc-2 hydrogen sulfide detecting apparatus 400) to ensure that when very alkaline industrial samples having a pH of 10 to 11, for example, are put into the reaction chamber 302, the test and buffer solution combination is still acidic, such that H₂S gas may be released. Preferably there is enough buffering capacity in the reaction chamber 302 to lower the pH capacity to below 4.0.

In preferred embodiments, the sinc-1 hydrogen sulfide detecting apparatus 300 is built and standardized on a five mil sample load, against 20 mils of buffer. For higher concentrated samples, reduced sample size may be used. In an industrial setting, for example, an industrial sample may typically run 200 H₂S parts per million. Instead of using a five mil load to detect the H₂S concentration, a one mil load of sample could be used. The resulting concentration determined would then be multiplied by 5 to reach the true concentration of the sample. For rich amines that run 8,000 to 12,000 ppm, only 0.1 mils, or one hundred microliters, need be used. The resulting concentration determined by the electrode would then be multiplied by 50 to calculate the true H₂S concentration of the sample.

For the lower concentration range, increasing the concentration allows the sinc-1 hydrogen sulfide detecting apparatus 300 to extend its measurements below 30 ppm. At the higher range, the dilution allows the measured concentration to fall back down into the standardized measurable range, so that by concentration and dilution the size of the range measured may be increased of from being just a static 34 to 260 parts per million, to from one part per million to 14,000 parts per million.

By adjusting the sample load volume, a peak that falls in the voltage range is achieved based on the H₂S that crossed and is trapped. This way, even though a largely different range of H₂S concentration of sample may be loaded, the sinc-1 hydrogen sulfide detecting apparatus 300 is standardized so that the amount of H₂S that is trapped in the trapping chamber 304 falls within a set range for reading. With a 5 ml sample, the range is standardized to 34 to 260 ppm.

Turning next to FIGS. 16-23, the sinc-2 hydrogen sulfide detecting apparatus 400 is shown. The sinc-2 hydrogen sulfide detecting apparatus 400 is similar to the sinc-1 hydrogen sulfide detecting apparatus 300 in design. The sinc-2 hydrogen sulfide detecting apparatus 400 has a reaction chamber 402, a trapping chamber 404 separated from the reaction chamber 402 by a permeable membrane 406, a deposit passage 408 to place test material into the reaction chamber 402, a testing passage 410 to access the trapping chamber 404, a fluid tight deposit cap 412 covering the deposit passage 408, and a fluid tight testing cap 414 covering the testing passage 410. The detecting apparatus preferably is formed from a base 416 and a lid 418 hermetically sealed together at a joint 420. One or more gaskets 422 and the membrane 406 are preferably retained between the lid 418 and the base 416 at or adjacent to the joint 420. The deposit passage 408 is defined by a bore in the base 416. The testing passage is defined by a bore in the lid 418. The base 416 preferably has a foot 424 at a bottom of the base 416 that extends around the base 416 to provide stability for the sinc-2 hydrogen sulfide detecting apparatus 400.

Inside the trapping chamber 404 is preferably 300 ul of 9.6 pH a tris buffer with 1 mM final concentration of MBB (monobromobimane). Inside of the reaction chamber 402 is 1.2-1.5 mls of 2.6 pH of a phosphate buffer containing 0.1 mM DPTA with/wo 1 mM TCEP (Tris (2-carboxyethyl) phosphine hydrochloride). The sinc-2 hydrogen sulfide detecting apparatus 400 accepts preferably a 10.0-50.0 microliter volume sample.

The reaction in the trapping chamber 404 of the sinc-2 hydrogen sulfide detecting apparatus 400 in one embodiment is MBV conversion to SVB, which fluoresces. Though the lid 418 and base 416 may be made out of clear material, making the lid 418 and base 416, or lid 418 and membrane 406 out of opaque material is an option to protect the fluorescent properties of the chemicals in the trapping chamber 404. With such opaqueness, the users would not be as concerned with working with the sinc-2 hydrogen sulfide detecting apparatus 400 it in the dark.

To use the sinc-2 hydrogen sulfide detecting apparatus 400, a user preferably holds the sinc-2 hydrogen sulfide detecting apparatus 400 such that the deposit passage 408 is oriented into the upwards direction. The user then removes the deposit cap 412 from the deposit passage 408, places a preferably biological sample into the reaction chamber 402, and replaces the deposit cap 412. The sample is then allowed to react with the buffer, and H₂S is liberated. The H₂S then migrates across the H₂S permeable membrane 406 from the reaction chamber 402 into the trapping chamber 404. In the alkaline conditions in the trapping chamber 404, the H₂S loses an H⁺ ion, and becomes HS⁻, a species which no longer freely permeates across the membrane 406. This allows the H₂S to be effectively trapped in the trapping chamber 404 and build up concentration.

After a given amount of time, the testing cap 414 is preferably removed, and a pipette is in inserted into the testing passage 410 and preferably into the trapping chamber 404. The pipette is used to remove the solution containing H₂S bound to fluorescent marker from the trapping chamber 404. The bound H₂S is then run on HPLC to determine concentration.

The trapping chamber 404 is preferably concave, with a conical peaked upper lid wall 426. This shape simultaneously reduces the volume of the trapping chamber compared to a cylinder shape, while both maintaining a larger surface area for the permeable membrane 406, and preserving sufficient height for the deposit passage 408 to access the trapping chamber 404, preferably large enough to allow a pipette to pass though. In a further embodiment, a slanted interior upper lid wall of the trapping chamber 404 is also an option. The preferred embodiment though is the concave shape shown in FIG. 17, as it gives more equal depth through the solution to the membrane than a slanted upper lid wall. If the trapping chamber gets too small air bubbles can form because of the smaller surface. Concave roof has an advantage that if an air bubble forms, it can roll up to the top of the concave peak.

The volume of the reaction chamber 402 for the sinc-2 hydrogen sulfide detecting apparatus 400 is preferably between 1.0 and 2.0 ml, more preferably between 1.25 and 1.75 ml, and most preferably 1.50 ml. The trapping chamber 404 of the sinc-2 hydrogen sulfide detecting apparatus 400 is preferably between 0.10 ml and 0.50 ml, more preferably between 0.2 ml and 0.4 ml, and most preferably 0.30 ml. Preferably there is four times the volume of buffer as to the sample to be added for the biologic testing apparatus. Preferably, the buffer solution is 0.07 molar.

While the preferred reading method for the sinc-1 hydrogen sulfide detecting apparatus 300 embodiment is electrochemical, the preferred reading method for the sinc-2 hydrogen sulfide detecting apparatus 400 embodiment is HPLC fluorescence.

The membrane for both sinc-1 and sinc-2 hydrogen sulfide detecting apparatuses 300, 400 is PDMS poly dimethylsiloxane that is between 0.62 to 100 microns in thickness, and has a pore size to allow passage of H₂S. Double sided thin fill adhesive to stick to bucket. Then gasket goes on the membrane and the lid goes on. Then the cap is hermetically sealed via sonic welding. Because of the size of the apparatus there are limits to the type of adhesion, mechanical or chemical, that can be used for the lid

Detection is currently in amines, biologics, and water. To use the device with hydrocarbons a different, hydrophobic and hydrocarbon fast membrane permeable to H2S and not HS1 may be used, such as a fluoropolymer like PTFE.

This embodiment of the sinc-2 hydrogen sulfide detecting apparatus 400 has many benefits, some of which are as follows. It shortens the workflow. The workflow with current technology involves putting a sample in test tubes, changing, putting different solutions on that, letting it go overtime. That is a complicated workflow with multiple pipetting steps, where the user can only do a limited number at a time. The disclosed hydrogen sulfide detecting apparatus 300, 400 allows a user to do much more. Also, importantly, current technology conducts test in open tubes, and while a test is being conducted in open tubes, H2S can be lost from the system, because it's labile. The disclosed hydrogen sulfide detecting apparatus 300, 400 preserve sample integrity, because once the sample is in the hydrogen sulfide detecting apparatus 300, 400, all the H2S is going to stay in the system.

The foregoing description and accompanying drawings illustrate the principles, exemplary embodiments, and modes of operation of the invention. However, the invention should not be construed as being limited to the particular embodiments discussed above. Many modifications of the embodiments described herein will come to mind to one skilled in the art having the benefit of the teaching presented in the foregoing descriptions and the associated drawings. Accordingly, it should be appreciated that variations to those embodiments can be made by those skilled in the art without departing from the scope of the invention.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A hydrogen sulfide (H₂S) detecting apparatus comprising:
a single reaction chamber defining a first volume;
a single trapping chamber positioned adjacent to the reaction chamber defining a second volume; and
an H₂S-permeable membrane positioned between and separating the reaction chamber and the of trapping chamber;
wherein the first volume is greater than the second volume.

2. The hydrogen sulfide (H₂S) detecting apparatus of claim 1 wherein the first volume being between 4 and 7 times as large as the second volume.

3. The hydrogen sulfide (H₂S) detecting apparatus of claim 1 wherein the reaction chamber is substantially defined by an interior of walls of a base and the membrane the trapping chamber is defined by an interior of walls of a lid and the member.

4. The hydrogen sulfide (H₂S) detecting apparatus of claim 3 further comprising a deposit passage to access and deposit a sample into the reaction chamber, the deposit passage one of extending from the walls of the base and being defined by a bore in the walls of the base.

5. The hydrogen sulfide (H₂S) detecting apparatus of claim 3 further comprising a testing passage to access the testing chamber, the testing passage one of extending from the walls of the lid and being defined by a bore in the walls of the lid.

6. The hydrogen sulfide (H₂S) detecting apparatus of claim 3 further comprising one of the base and the lid, the lid and the membrane, and the base, the lid, and the membrane being opaque.

7. The hydrogen sulfide (H₂S) detecting apparatus of claim 3 further comprising the lid and the base being hermetically sealed to one another.

8. The hydrogen sulfide (H₂S) detecting apparatus of claim 7 wherein the lid and the base are sonically sealed to one another.

9. The hydrogen sulfide (H₂S) detecting apparatus of claim 3 further comprising one or more feet extending from the base to stabilize the apparatus.

10. The hydrogen sulfide (H₂S) detecting apparatus of claim 4 further comprising one or more feet extending from the base to stabilize the apparatus wherein the feet are oriented on an opposite side of the apparatus from the deposit passage.

11. The hydrogen sulfide (H₂S) detecting apparatus of claim 3 further comprising one of a fluid tight deposit cap removably located in and sealing off a deposit passage, a fluid tight testing cap removably located in and sealing off a testing passage, and both a testing cap and a deposit cap.

12. The hydrogen sulfide (H₂S) detecting apparatus of claim 1 wherein the reaction chamber is preloaded with a buffer to make the reaction chamber environment acidic, with a pH below 6.

13. The hydrogen sulfide (H₂S) detecting apparatus of claim 1 wherein the trapping chamber is preloaded with a buffer to make the trapping chamber environment basic, with a pH above 8.

14. The hydrogen sulfide (H₂S) detecting apparatus of claim 3, wherein an inner wall of the lid is concave and forms a conical recess into the inner wall of the lid, and the tip of the conical recess is circumferentially aligned with a center of the H₂S permeable membrane.

15. The hydrogen sulfide (H₂S) detecting apparatus of claim 1 wherein a fluorescent chemical that binds to HS⁻ is preloaded into the trapping chamber.

16. The hydrogen sulfide (H₂S) detecting apparatus of claim 1 wherein the membrane is permeable to H₂S, but substantially impermeable to HS−.

17. The hydrogen sulfide (H₂S) detecting apparatus of claim 1 wherein the trapping chamber contains a pH above 9 of a Tris base buffer containing one of 0.1 mM DPTA (Diethylenetriaminepentaacetic acid) and MBB (monobromobimane), and the reaction chamber contains a pH below 3 of a phosphate buffer containing 0.1 mM DPTA.

18. The hydrogen sulfide (H₂S) detecting apparatus of claim 17 wherein the reaction chamber further contains 1 mM TCEP (Tris (2-carboxyethyl) phosphine hydrochloride).

19. The hydrogen sulfide (H₂S) detecting apparatus of claim 3, wherein the lid defines an elevated spacing and a deposit passage extends substantially orthogonally to a plan defined by the membrane.

20. A hydrogen sulfide (H₂S) detecting apparatus comprising:
a single reaction chamber defining a first volume;
a single trapping chamber positioned adjacent to the reaction chamber defining a second volume;
an H₂S-permeable membrane positioned between and separating the reaction chamber and the of trapping chamber;
the reaction chamber being substantially defined by an interior of walls of a base and the membrane;
the trapping chamber being defined by an interior of walls of a lid and the member;
a testing passage to access the testing chamber, the testing passage one of extending from the walls of the lid and being defined by a bore in the walls of the lid;
one of the base and the lid, the lid and the membrane, and the base, the lid, and the membrane being opaque;
the lid and the base being sonically welded and hermetically sealed to one another;
one or more feet extending from the base to stabilize the apparatus;
wherein
the first volume is greater than the second volume;
the first volume being between 5 and 6 times as large as the second volume;
trapping chamber contains a pH above 9 of a Tris base buffer containing one of 0.1 mM DPTA (Diethylenetriaminepentaacetic acid) and MBB (monobromobimane); and
the reaction chamber contains a pH below 3 of a phosphate buffer containing 0.1 mM DPTA.
